(19) United States
(12) Patent Application Publication
(10) Pub. No.: US 2024/0038083 A1
(43) Pub. Date: Feb. 1, 2024

(54) PUBLICITY-EDUCATION PUSHING METHOD AND SYSTEM BASED ON MULTI-SOURCE INFORMATION FUSION

(71) Applicant: ZHEJIANG LAB, Hangzhou (CN)

(72) Inventors: Jingsong LI, Hangzhou (CN); Huiyao SUN, Hangzhou (CN); Tianshu ZHOU, Hangzhou (CN); Yu TIAN, Hangzhou (CN); Ying ZHANG, Hangzhou (CN)

(21) Appl. No.: 18/360,832

(22) Filed: Jul. 28, 2023

(30) Foreign Application Priority Data
Jul. 29, 2022 (CN) 202210902893.3

(51) Int. Cl. G09B 5/02; G16H 50/20; G16H 20/10; G06N 20/00

(52) U.S. Cl. CPC G09B 5/02; G16H 50/20; G16H 20/10; G06N 20/00; G16H 10/60

(57) ABSTRACT

The present disclosure discloses a publicity-education pushing method and system based on a multi-source information fusion. The method includes: step S1: constructing a patient publicity-education knowledge graph, and pushing the patient publicity-education knowledge graph to a patient through a publicity-education applet; step S2: fusing and correcting patient basic information, patient diagnosis-treatment information, patient eye movement information and a patient personality inventory to obtain patient multi-source information; step S3: constructing a compliance prediction model through a neural network by using the patient multi-source information and collected patient medication taking behavior data; and step S5: building a system rule base, and after searching for a corresponding disease and treatment in the patient publicity-education knowledge graph through information returned by the system rule base, pushing the disease and the treatment to the patient through the publicity-education applet.

### CROSS-REFERENCE TO RELATED APPLICATIONS

The present application claims priority to Chinese Patent Application No. 202210902893.3, filed on Jul. 29, 2022, the content of which is incorporated herein by reference in its entirety.

### TECHNICAL FIELD

The present disclosure relates to the technical field of data collection, in particular to a publicity-education pushing method and a system based on a multi-source information fusion.

### BACKGROUND

In recent years, more and more pharmaceutical companies and hospitals have begun to attach great importance to patient education. Patient education is not only about conveying information superficially, but also about improving patient compliance through highly professional knowledge, accurate medical information, as well as medical knowledge that cannot be found online. During the actual treatment process, due to reasons of increasing age of patients, a lower education level, inattention to diet by the patients, negative attitudes toward adverse reactions, unclear understanding of the diseases, etc., poor patient compliance is caused, and the therapeutic effect of the disease and the patient's quality of life are seriously affected. Therefore, the patient education plays a crucial role and has significant social significance in disease treatment.

Eye movement tracking mainly describes dynamic changes of pupils by automatically detecting relative positions of the pupils of human eyes or estimating relative positions of sight directions, so as to intuitively reflect fixation points and fixation time of the human. The main methods include fixation, saccades, smooth tracking movement, blinking, and eyeball vibration. In daily life, most of the information is obtained through fixation. When central foveae of the human eyes are aligned with an object to be observed for more than 100 ms, the observed object can be fully processed on the central foveae to form a clear image. Currently, eye movement tracking methods may be classified into four types: search coil recording method, infrared method, current recording method, and video recording method.

In the current research, patient education is mostly carried out by patient education specialists or nursing staff for hospitalized patients, including issuing health education manuals, education and publicity boards, face-to-face preaching, communicating with the patients, telling the patients about daily diet and other methods. These traditional methods have the following shortcomings: (1) patient education can only be carried out for the hospitalized patients. For patients who need long-term treatment due to chronic diseases or chemotherapy, they need to take medicine by themselves or go to the hospital on time for treatment, and there are some reasons such as forgetting treatment, an unsatisfactory effect after treatment and fear of side effects, which lead to unsatisfactory compliance. For the existing patient education, it is difficult to improve the compliance of such patients, and improving the compliance of such patients can better alleviate the symptoms of the patients and improve their quality of life. (2) It costs a lot to educate the patients without difference. On the one hand, a large number of patient education specialists or nursing staff is required, and at the same time, the patient education specialists or nursing staff need to have strong medical related knowledge and psychological knowledge. After understanding a patient's personal diagnosis and treatment scheme, the patient education specialists or nursing staff need to have a deep understanding for the disease, side effects of treatment and other knowledge, and not only can unilaterally preach to the patients, but also can answer all kinds of problems from the patients; and on the other hand, there is no way to provide different education for patients based on their differences. Some patients do not need too much patient education, and only need a corresponding disease manual; while some patients require a lot of attention and answers with professional knowledge, especially older and less educated patients. However, patients are usually unable to be accurately distinguished. At the same time, for some patients with negative emotions towards disease treatment, additional psychological comfort and positive guidance are needed.

In view of this, the present disclosure provides a publicity-education pushing method and a system based on a multi-source information fusion to solve the above technical problems.

### SUMMARY

In order to solve the above technical problems, the present disclosure provides a publicity-education pushing method and system based on a multi-source information fusion.

The present disclosure adopts a technical solution as follows:
a publicity-education pushing method based on a multi-source information fusion includes the following steps:
step S1: constructing a patient publicity-education knowledge graph through public knowledge, a clinical expert supplement and an electronic medical record, and pushing the patient publicity-education knowledge graph to a patient through a publicity-education applet;
step S2: collecting patient basic information and patient diagnosis-treatment information through the electronic medical record, collecting patient eye movement information and a patient personality inventory through the publicity-education applet, and fusing and correcting the patient basic information, the patient diagnosis-treatment information, the patient eye movement information and the patient personality inventory to obtain patient multi-source information;
step S3: constructing a compliance prediction model through a neural network by using the patient multi-source information and data collected on patient medication-taking behavior;
step S4: predicting a patient category by using the compliance prediction model to obtain a patient classification; and
step S5: building a system rule base by using the patient multi-source information, and after searching for a corresponding disease and treatment in the patient publicity-education knowledge graph through feedback information from the system rule base, pushing the disease and the treatment to the patient through the publicity-education applet.

Furthermore, in step S1:
the public knowledge is a patient treatment method, and an adverse reaction and/or an indication to the treatment method collected from a public knowledge base, an electronic version of guide and/or a disease form;
the clinical expert supplement is a supplement and refinement to incompleteness of the disease and/or incompleteness of a disease treatment mode performed through clinical experience of a clinician and/or a relevant expert; and
the electronic medical record includes clinical data information of electronic medical records from a plurality of medical institutions.

Furthermore, in step S1, storage of the patient publicity-education knowledge graph represents a triad in the form of <subject, predicate, object> through an RDF structure, and finally the patient publicity-education knowledge graph with the disease as the subject, the adverse reaction, surgical treatment and/or drug treatment as the predicate, and a value pointed by the predicate as the object is formed; and a storage structure of the patient publicity-education knowledge graph is presented in a multimodal manner, including text, and a text-related picture and/or video.

Furthermore, step S2 specifically includes following sub-steps:
step S21: collecting the patient basic information and the patient diagnosis-treatment information through the electronic medical record, the patient basic information includes fields corresponding to the following parameters: a patient identity card number, a patient ID, an age, an education level, a geographical factor and/or accompanying status of family members, and the patient diagnosis-treatment information includes fields corresponding to the following parameters: visit datetime, the disease, a treatment mode, the drug treatment and/or the surgical treatment; and fusing the patient basic information and the patient diagnosis-treatment information to obtain electronic medical record information, with the electronic medical record information corresponding to a latest visit datetime as valid electronic medical record information;
step S22: sending the patient publicity-education knowledge graph corresponding to the disease to the patient by the publicity-education applet, collecting the patient eye movement information and the patient personality inventory through the publicity-education applet, and fusing the patient eye movement information and the patient personality inventory through the patient identity card number to obtain publicity-education applet information, with the publicity-education applet information corresponding to a latest video datetime as valid publicity-education applet information;
step S23: fusing the valid electronic medical record information and the valid publicity-education applet information through the patient identity card number to obtain the patient multi-source information; and
step S24: identifying whether the electronic medical record information corresponding to the latest visit datetime in the electronic medical record information is consistent with electronic medical record information of the valid electronic medical record information, identifying whether the publicity-education applet information corresponding to the latest video datetime is consistent with publicity-education applet information of the valid publicity-education applet information; if at least one of the two identification results represents inconsistency, repeating step S21 to step S23 for re-fusion until both identification results represent consistency to complete a correction of the patient multi-source information.

Furthermore, in step S22, the patient eye movement information includes detention page content, an average fixation time, a number of fixations, a fixation order, an average saccade amplitude, a number of saccades, a scanning duration, and/or a scanning direction; and the patient personality inventory includes openness, conscientiousness, extraversion, agreeableness, emotional stability, and/or non-personality-inventory data without actively performing personality inventory.

Furthermore, step S3 specifically includes the following sub-steps:
step S31: collecting the data on patient medication-taking behavior, the data on patient medication-taking behavior being divided into full compliance, partial compliance and full non-compliance, and using the patient multi-source information and the data on patient medication-taking behavior as training data for a model; and
step S32: training a neural network model using the training data, outputting a result by adopting a Sigmoid activation function, obtaining different models by calculating macros of prediction data with the macros between 0 and 1 and continuously changing training parameters, and finally selecting a prediction model with a highest macro value as the compliance prediction model.

Furthermore, step S5 specifically includes the following sub-steps:
step S51: using a feature vector of the eye movement information in the patient multi-source information as an input and a state of the eye movement information as a feedback, establishing rules through the input and the feedback, and forming the system rule base by the plurality of rules; and
step S52: inputting the feature vector of the eye movement information to the system rule base, and after searching for the corresponding disease and treatment in the patient publicity-education knowledge graph through content and a form returned by the system rule base, pushing content of an adverse reaction and a corresponding video obtained by searching to the patient through the publicity-education applet.

Furthermore, in step S5, the predicate of the patient publicity-education knowledge graph is returned according to patient information, and searching and pushing are performed through the patient publicity-education knowledge graph.

Furthermore, in step S5, when it is found that the patient is more receptive to a picture or a video through eye movement data, pictures and videos of pushing content and a pushing form are further searched for and pushed through a determine of an automatic pushing system on the pushing content and the pushing form.

The present disclosure further provides a publicity-education pushing system based on a multi-source information fusion, including:
a patient publicity-education knowledge graph module, configured to push a patient publicity-education knowledge graph to a patient for publicity and education;
a patient multi-source information fusion module, configured to collect patient basic information and patient diagnosis-treatment information through an electronic medical record, collect patient eye movement information and a patient personality inventory through a publicity-education applet, and fuse and correct the patient basic information, the patient diagnosis-treatment information, the patient eye movement information and the patient personality inventory to obtain patient multi-source information;
a compliance prediction module, configured to construct a compliance prediction model through a neural network by using the patient multi-source information and data collected on patient medication-taking behavior, and obtain a patient classification by a prediction through the compliance prediction model; and
an automatic pushing system module, configured to build a system rule base by using the patient multi-source information, and after searching for a corresponding disease and treatment in the patient publicity-education knowledge graph through feedback information from the system rule base, push the disease and the treatment to the patient through the publicity-education applet.

The present disclosure has beneficial effects that:

1. The present disclosure abandons an original mode of education only for hospitalized patients, and can effectively educate the patients through applets or wearable devices under the condition that the existing Internet and the Internet of things are developed. In this way, it can not only save a large number of patient education specialists or nursing staff, but also help patients who are not hospitalized and take medications for a long time to improve their understanding of medications. The present disclosure pushes patient education content or establishes real-time communication with professionals through an applet, and when the patients are confused or need help, the patients can get answers through text, pictures, videos, voices and other means.

2. In order to solve the problem of undifferentiated patient education of the patients and cost reduction, the patients are classified by collecting multi-source information of the patients. In order to avoid wasting more costs on patients who do not need too much patient education, and meanwhile prevent these patients from having “boredom” psychology, patients who do not have manpower or time to invest in patient education need to be classified. Not only the basic information and diagnosis and treatment information of the patients are collected, but also the personality inventory of the patients and eye movement data when watching the pushed education content are collected, and the eye movement data mainly refer to eye movement features captured during detention at a page, including an average fixation time, a number of fixations, a fixation order, an average saccade amplitude, a number of saccades, a scanning duration, a scanning direction, etc.

3. In order to solve the problem of requiring a large number of patient education specialists or nursing staff, the knowledge graph is used to store publicity-education knowledge, and relevant information is automatically pushed after the patients are distinguished. The knowledge graph stores various forms of content required by publicity-education interference, storage forms are mainly divided into three: text, pictures and videos, the content mainly includes education manuals, adverse reactions, indications, applicable diseases, side effects, harms without treatment, etc., and the education manuals are basic knowledge of the disease, common treatment methods and other basic information. The knowledge graph is used to be able to store a large amount of information and various forms, instead of manual knowledge storage, more accurate, efficient, professional, easy to search and apply.

### BRIEF DESCRIPTION OF DRAWINGS

FIG. 1 is a flow diagram of a publicity-education pushing method based on a multi-source information fusion of the present disclosure;

FIG. 2 is a structural diagram of a publicity-education pushing system based on a multi-source information fusion of the present disclosure;

FIG. 3 is a schematic diagram of a patient publicity-education knowledge graph of an embodiment of the present disclosure;

FIG. 4 is a schematic diagram of a patient multi-source information fusion of an embodiment of the present disclosure; and

FIG. 5 is a schematic diagram of a compliance prediction model of an embodiment of the present disclosure.

### DESCRIPTION OF EMBODIMENTS

The following description of at least one exemplary embodiment is in fact illustrative only and never acts as any limitation on the present disclosure and its application or use. Based on the embodiments of the present disclosure, all other embodiments obtained by those ordinarily skilled in the art without creative labor fall within the scope of protection of the present disclosure.

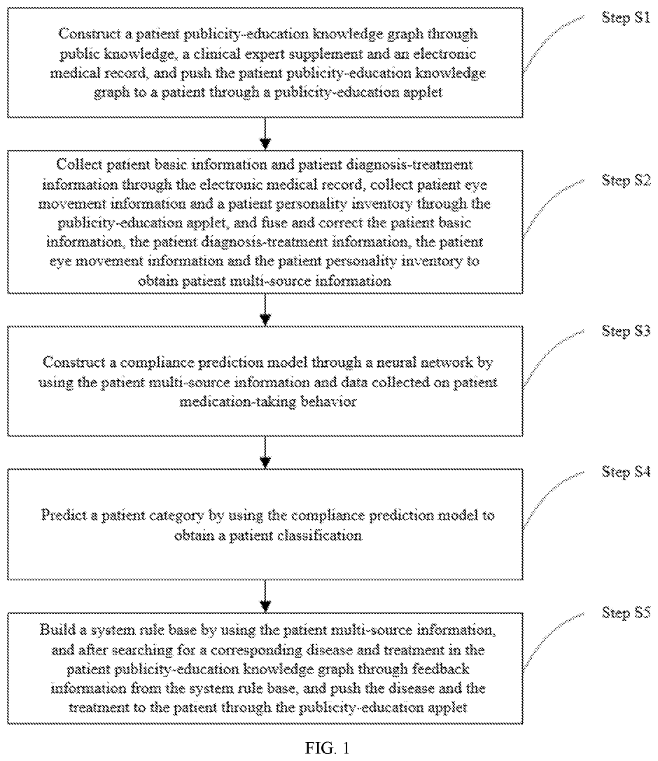

Referring to FIG. 1, a publicity-education pushing method based on a multi-source information fusion includes following steps:
step S1: a patient publicity-education knowledge graph is constructed through public knowledge, a clinical expert supplement and an electronic medical record, and the patient publicity-education knowledge graph is pushed to a patient through a publicity-education applet;
the public knowledge is a patient treatment method, and an adverse reaction and/or an indication to the treatment method collected from a public knowledge base, an electronic version of guide and/or a disease form;
the clinical expert supplement is a supplement and refinement to incompleteness of the disease and/or incompleteness of a disease treatment mode performed through clinical experience of a clinician and/or a relevant expert;
the electronic medical record includes clinical data information from electronic medical records from a plurality of medical institutions; and
storage of the patient publicity-education knowledge graph represents a triad in the form of <subject, predicate, object> through an RDF structure, and finally the patient publicity-education knowledge graph with the disease as the subject, an adverse reaction, surgical treatment and/or drug treatment as the predicate, and a value pointed by the predicate as the object is formed; and a storage structure of the patient publicity-education knowledge graph is presented in a multimodal manner, including text, and a text-related picture and/or video.
Step S2: patient basic information and patient diagnosis-treatment information are collected through the electronic medical record, patient eye movement information and a patient personality inventory are collected through the publicity-education applet, and the patient basic information, the patient diagnosis-treatment information, the patient eye movement information and the patient personality inventory are fused and corrected to obtain patient multi-source information;
step S21: the patient basic information and the patient diagnosis-treatment information are collected through the electronic medical record, the patient basic information includes fields corresponding to the following parameters: a patient identity card number, a patient ID, an age, an education level, a geographical factor and/or accompanying status of family members, and the patient diagnosis-treatment information includes fields corresponding to the following parameters: visit datetime, the disease, a treatment mode, a drug treatment and/or a surgical treatment; and the patient basic information and the patient diagnosis-treatment information are fused to obtain electronic medical record information, and the electronic medical record information corresponding to a latest visit datetime is used as valid electronic medical record information;
step S22: the publicity-education applet sends the patient publicity-education knowledge graph corresponding to the disease to the patient, the patient eye movement information and the patient personality inventory are collected through the publicity-education applet, the patient eye movement information and the patient personality inventory are fused through the patient identity card number to obtain publicity-education applet information, and the publicity-education applet information corresponding to a latest video datetime is used as valid publicity-education applet information;
the patient eye movement information includes detention page content, an average fixation time, a number of fixations, a fixation order, an average saccade amplitude, a number of saccades, a scanning duration, and/or a scanning direction; and the patient personality inventory includes openness, conscientiousness, extraversion, agreeableness, emotional stability, and/or non-personality-inventory data without actively performing personality inventory;
step S23: the valid electronic medical record information and the valid publicity-education applet information are fused through the patient identity card number to obtain the patient multi-source information; and
step S24: whether the electronic medical record information corresponding to the latest visit datetime in the electronic medical record information is consistent with electronic medical record information of the valid electronic medical record information is identified, whether the publicity-education applet information corresponding to the latest video datetime is consistent with publicity-education applet information of the valid publicity-education applet information is identified, and if at least one of the two identification results represents inconsistency, step S21 to step S23 are repeated for re-fusion until both are consistent to complete a correction of the patient multi-source information.
Step S3: a compliance prediction model is constructed through a neural network by using the patient multi-source information and data collected on patient medication-taking behavior;
step S31: the data on patient medication-taking behavior are collected, the data on patient medication-taking behavior are divided into full compliance, partial compliance and full non-compliance, and the patient multi-source information and the data on patient medication-taking behavior are used as training data for a model; and
step S32: a neural network model is trained using the training data, a result is output by adopting a Sigmoid activation function, different models are obtained by calculating macros of prediction data with the macros between 0 and 1 and continuously changing training parameters, and a prediction model with a highest macro value is finally selected as the compliance prediction model.
Step S4: a patient category is predicted by using the compliance prediction model to obtain a patient classification;
step S5: a system rule base is built by using the patient multi-source information, and after searching for a corresponding disease and treatment in the patient publicity-education knowledge graph through feedback information from the system rule base, the disease and the treatment are pushed to the patient through the publicity-education applet;
step S51: a feature vector of the eye movement information in the patient multi-source information is used as an input, a state of the eye movement information is used as a feedback, rules are established through the input and the feedback, and the system rule base is formed by the plurality of rules; and
step S52: the feature vector of the eye movement information is input to the system rule base, and after searching for the corresponding disease and treatment in the patient publicity-education knowledge graph through content and a form returned by the system rule base, content of an adverse reaction and a corresponding video obtained by searching are pushed to the patient through the publicity-education applet;
the predicate of the patient publicity-education knowledge graph is returned according to patient information, and searching and pushing are performed through the patient publicity-education knowledge graph; and
when it is found that the patient is more receptive to a picture or a video through eye movement data, pictures and videos of pushing content and a pushing form are further searched for and pushed through a determine of an automatic pushing system on the pushing content and the pushing form.

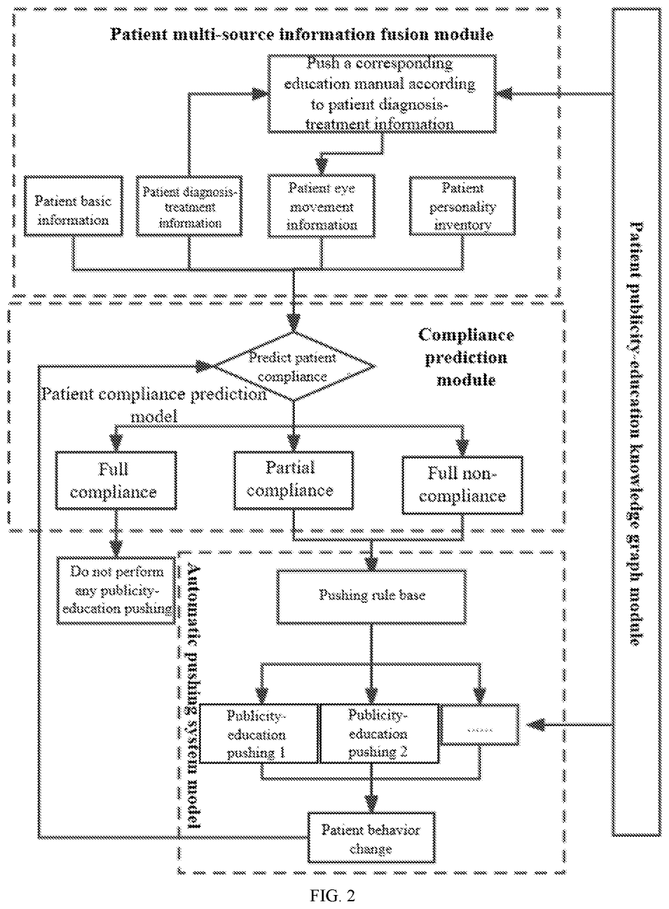

Referring to FIG. 2, a publicity-education pushing system based on a multi-source information fusion includes:
a patient publicity-education knowledge graph module, configured to push a patient publicity-education knowledge graph to a patient for publicity and education;
a patient multi-source information fusion module, configured to collect patient basic information and patient diagnosis-treatment information through an electronic medical record, collect patient eye movement information and a patient personality inventory through a publicity-education applet, and fuse and correct the patient basic information, the patient diagnosis-treatment information, the patient eye movement information and the patient personality inventory to obtain patient multi-source information;
a compliance prediction module, configured to construct a compliance prediction model through a neural network by using the patient multi-source information and data collected on patient medication-taking behavior, and obtain a patient classification by a prediction through the compliance prediction model; and
an automatic pushing system module, configured to build a system rule base by using the patient multi-source information, and after searching for a corresponding disease and treatment in the patient publicity-education knowledge graph through feedback information from the system rule base, push the disease and the treatment to the patient through the publicity-education applet.

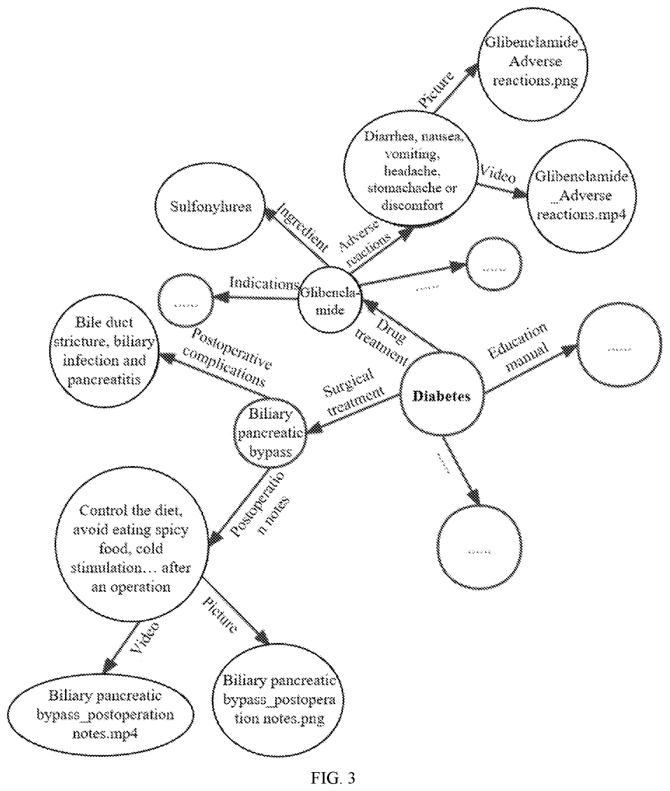

Embodiment:
step S1: a patient publicity-education knowledge graph is constructed through public knowledge, a clinical expert supplement and an electronic medical record, and the patient publicity-education knowledge graph is pushed to a patient through a publicity-education applet;
the patient publicity-education knowledge graph is mainly used as a knowledge support of a patient pushing education manual and an automatic pushing system module, helps patients to understand effective knowledge more accurately and comprehensively, and meanwhile helps professionals to quickly search for effective answers in one-to-one answers, which is more professional and accurate. Knowledge in the construction of the patient publicity-education knowledge graph is obtained through three following processes:
the public knowledge is a patient treatment method, and an adverse reaction and/or an indication to the treatment method collected from a public knowledge base, an electronic version of guide and/or a disease form;
the clinical expert supplement is a supplement and refinement to incompleteness of the disease and/or incompleteness of a disease treatment mode performed through clinical experience of a clinician and/or a relevant expert;
the electronic medical record includes clinical data information of electronic medical records from a plurality of medical institutions; and
referring to FIG. 3, storage of the patient publicity-education knowledge graph represents a triad in the form of <subject, predicate, object> through an RDF structure, and finally the patient publicity-education knowledge graph with the disease as the subject, an adverse reaction, the surgical treatment and/or the drug treatment as the predicate, and a value pointed by the predicate as the object is formed; and a storage structure of the patient publicity-education knowledge graph is presented in a multimodal manner, including text, and a text-related picture and/or video.

The storage of the patient publicity-education knowledge graph is represented by the RDF structure, basic component units of which are facts, and each fact is represented as a triad shaped like <subject, predicate, object>. The subject is usually any one of entities, facts, or concepts; the predicate is usually a relation or attribute; and the object may be entities, events and concepts, and may also be ordinary values. In the patient publicity-education knowledge graph, the disease is used as the subject, an adverse reaction, the surgical treatment, the drug treatment and other relations are used as the predicate, and a value corresponding to the relation pointed to by the predicate is used as the object. For example, <diabetes, surgical treatment, biliary pancreatic bypass>, <diabetes, drug treatment, glibenclamide>, <biliary pancreatic bypass, postoperative complications, bile duct stricture, biliary infection, pancreatitis> etc. are used to represent all information of diabetes diseases.

A storage structure of the patient publicity-education knowledge graph is presented in a multimodal manner, a storage content of the patient publicity-education knowledge graph is not only limited to text and further includes a text-related picture and/or video, for the picture and the video, when the triad is stored, the triad is stored in a form of a picture name and a video name, when the patient publicity-education knowledge graph is searched, a name corresponding to the picture or video is searched for through the predicate equal to the picture or video, and the corresponding picture and video are retrieved from a repository according to the name.

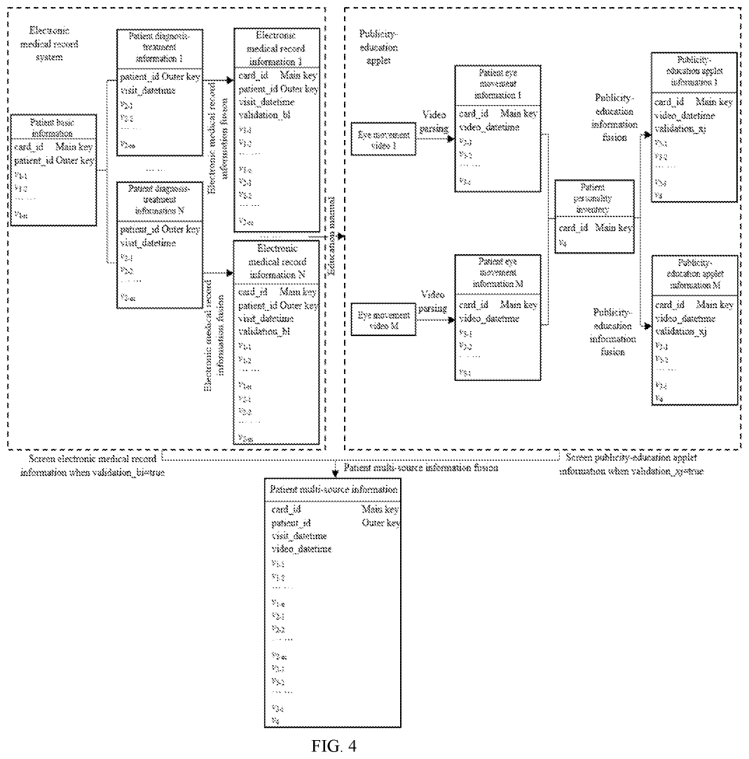

Referring to FIG. 4, step S2: patient basic information and patient diagnosis-treatment information are collected through the electronic medical record, patient eye movement information and a patient personality inventory are collected through the publicity-education applet, and the patient basic information, the patient diagnosis-treatment information, the patient eye movement information and the patient personality inventory are fused and corrected to obtain patient multi-source information;
step S21: the patient basic information and the patient diagnosis-treatment information are collected through the electronic medical record, the patient basic information includes fields corresponding to the following parameters: a patient identity card number, a patient ID, an age, an education level, a geographical factor and/or accompanying status of family members, and the patient diagnosis-treatment information includes fields corresponding to the following parameters: visit datetime, the disease, a treatment mode, a drug treatment and/or a surgical treatment; and the patient basic information and the patient diagnosis-treatment information are fused to obtain electronic medical record information, and the electronic medical record information corresponding to a latest visit datetime is used as valid electronic medical record information; and
in an electronic medical record storage structure, one patient only stores one piece of patient basic information, including the patient identity card number (denoted as card_id) and the patient ID (denoted as patient_id), the present disclosure uses the patient ID (patient_id) as an outer key and the patient identity card number (card_id) as a main key, and other information of the patient basic information includes the age, the education level, the geographical factor, the family accompanying, etc., denoted as v_1={v_(1−1),v_(1−2), . . . , v_(1−n}; one patient will have a plurality of times of diagnosis-treatment information, each time of visit, hospitalization or physical examination will produce one piece of patient diagnosis-treatment information, it is assumed that the patient has N times of diagnosis-treatment information totally, in each time of patient diagnosis-treatment information, in addition to the patient ID (patient_id) as the outer key and the patient basic information, visit datetime (denoted as visit_datetime) is used as a timestamp, and the other including disease, treatment mode, drug treatment, surgical treatment, etc. are denoted as v_2={v_(2−1), v_(2−2), . . . , v_(2−m)}. When the patient basic information and N times of diagnosis-treatment information are fused respectively, there are N pieces of electronic medical record information, denoted as {card_id, patient_id, visit_datetime, video_datetime, [symbol] v_(1−1), v_(1−2), . . . , v_(1−n), v [symbol] _(2−1), v_(2−2), . . . , v_(2−m,) v_(3−1,) v_(3−2,), . . . , v_(3−1,) v_(4,)}, medical record valid information (denoted as validation_bl) judges whether the electronic medical record information is valid from the N pieces of electronic medical record information, only in the electronic medical record information of a latest visit datetime (visit_datetime), validation_bl=true, and in other electronic medical record information, validation_bl=false.

Step S22: the publicity-education applet sends the patient publicity-education knowledge graph corresponding to the disease to the patient, the patient eye movement information and the patient personality inventory are collected through the publicity-education applet, the patient eye movement information and the patient personality inventory are fused through the patient_identity card number to obtain publicity-education applet information, and the publicity-education applet information corresponding to a latest video datetime is used as valid publicity-education applet information; and
the patient eye movement information includes detention page content, an average fixation time, a number of fixations, a fixation order, an average saccade amplitude, a number of saccades, a scanning duration, and/or a scanning direction; and the patient personality inventory includes openness, conscientiousness, extraversion, agreeableness, emotional stability, and/or non-personality-inventory data without actively performing personality inventory.

The publicity-education applet uses the patient_identity card number (denoted as card_id) as the main key that is a patient unique identifier, the publicity-education applet sends the patient publicity-education map corresponding to the disease according to the patient identity card number (card_id) in the diagnosis-treatment information and disease information when validation_bl=true, when the patient checks the patient publicity-education map in the publicity-education applet, the publicity-education applet collects an eye movement video of the patient, and patient eye movement information is obtained through video analysis. One patient has the eye movement video of opening the publicity-education applet for a plurality of times to watch the patient publicity-education map, it is assumed that there are M times of opening the patient publicity-education map to collect the eye movement video, there are M pieces of patient eye movement information, collection video datetime (denoted as video_datetime) is used as a timestamp, and in addition to the patient identity card number (card_id) and the video datetime (video_datetime), other patient eye movement information of the publicity-education applet includes detention page content, an average fixation time, a number of fixations, a fixation order, an average saccade amplitude, a number of saccades, a scanning duration, a scanning direction, etc., denoted as v_3={v_(3−1), v_(3−2), . . . , v_(3−l)}; a patient personality inventory questionnaire is pushed in the publicity-education applet, and when the patient answers the patient personality inventory through the questionnaire, in addition to recording the patient identity card number (card_id), a patient personality inventory result is recorded, that is “openness”, “conscientiousness”, “personality inventory” “agreeableness”, “emotional stability”, or “non-personality-inventory data” without actively performing personality inventory, denoted as v_4. The M pieces of patient eye movement information in the publicity-education applet and the patient personality inventory are fused through the patient identity card number (card_id) to obtain M pieces of publicity-education applet information, and each piece of publicity-education applet information is detonated as {card_id, video_datetime, validation_xj, v_(3−1), v_(3−2), . . . , v_(3−l), v_4, publicity-education valid information (denoted as validation_xj) is to judge whether the publicity-education applet information is valid from the M pieces of publicity-education applet information, only in the publicity-education applet information of a latest video datetime (video_datetime), validation_xj=true, and in other pieces of publicity-education applet information, validation_xj=false.

Step S23: the valid electronic medical record information and the valid publicity-education applet information are fused through the patient identity card number to obtain the patient multi-source information;
the electronic medical record information with validation_bl=true is selected from N pieces of electronic medical record information, the publicity-education applet information of the validation_xj=true is selected from M pieces of publicity-education applet information, and two pieces of information are fused through the patient identity card number (card_id) to obtain the patient multi-source information, denoted as {card_id, patient_id, visit_datetime, video_datetime, v_(1−1), v_(1−2), . . . , v_(1−n), v_(2−1), v_(2−2), . . . , v_(2−m), v_(3−1), v_(3−2), . . . , v_(3−l), v_4}.

Step S24: whether the electronic medical record information corresponding to the latest visit datetime in the electronic medical record information is consistent with electronic medical record information of the valid electronic medical record information is identified, whether the publicity-education applet information corresponding to the latest video datetime is consistent with publicity-education applet information of the valid publicity-education applet information is identified, and if at least one of the two identification results represents inconsistency, step S21 to step S23 are repeated for re-fusion until both identification results represent consistency to complete a correction of the patient multi-source information.

The correction of the patient multi-source information occurs when the patient diagnosis-treatment information is added or new patient eye movement information is collected. According to a fusion process of the electronic medical record information, when the patient has a new visit, that is, one time of patient diagnosis-treatment information is added and is denoted as N+1, its visit datetime (visit_datetime) is latest time in previous visits, therefore, when the electronic medical record information is fused, validation_bl=true in the previous electronic medical record information is changed to validation_bl=false, and in the new electronic medical record information generated at the N+1 ^{th} time, validation_bl=true; and according to a fusion process of the publicity-education applet, when the patient receives a new patient publicity-education knowledge graph or checks the original patient publicity-education knowledge graph once again, one time of patient eye movement information is added and is denoted as M+1, its video datetime (video_datetime) is latest time in previous visits, therefore, when the publicity-education applet information is fused, validation_xj=true in the previous publicity-education applet information is changed to validation_xj=false, and in the new publicity-education applet information generated at the M+1 ^{th} time, validation_bl=true. Changes of validation_bl in the electronic medical record information and validation_xj in the publicity-education applet information require the correction of the patient multi-source information.

Firstly, whether the visit datetime (visit_datetime) in the original patient multi-source information is consistent with the visit datetime (visit_datetime) in the electronic medical record information of validation_bl=true is identified; and then, whether the video datetime (video_datetime) in the original patient multi-source information is consistent with the video datetime (video_datetime) in the publicity-education applet information of validation_xj=true is identified. If one or more of the two are inconsistent, a correction is performed. The changed patient identity card number (card_id) is identified, patient_identity card number (card_id) information in the patient multi-source information is deleted, and re-fusion is performed according to the above fusion process. After the patient multi-source information is corrected, the patient compliance prediction model is re-predicted as new data sub sequently.

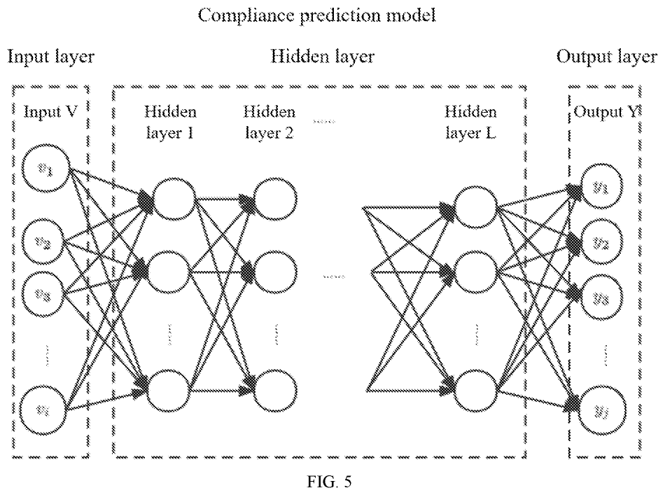

Referring to FIG. 5, step S3: a compliance prediction model is constructed through a neural network by using the patient multi-source information and data collected on patient medication-taking behavior;
step S31: the data on patient medication-taking behavior are collected, the data on patient medication-taking behavior are divided into full compliance, partial compliance and full non-compliance, and the patient multi-source information and the data on patient medication-taking behavior are used as training data for a model;
through the fusion process of the patient multi-source information, the patient multi-source information is obtained and represented as {card_id, patient_id, visit_datetime, video_datetime, v_(1−1), v_(1−2), . . . , v_(1−n), v_(2−1), v_(2−2), . . . , v_(2−m), v_(3−1), v_(3−2), . . . , v_(3−1), v_4}, according to the above patient basic information, the patient diagnosis-treatment information, the patient eye movement information and the patient personality inventory, respectively denoted as v_1, v_2 , v_3 , and v_4 , the patient multi-source information is represented as {card_id, patient_id, visit_datetime, video_datetime, v_1, v_2, v_3, v_4}, information in addition to the main key, the outer key and the time information is used as a feature vector of the patient multi-source information, denoted as V, and V={v_1, v_2, v_3, v_4}. In the training data, in addition to collecting the above information, medication taking behaviors of 1000 patients are randomly collected through smart bracelet wearing or applet manual filling so as to be judged as three categories of full compliance, partial compliance (exceeding or short of doses of medication, increasing or decreasing the number of times of medication taking, etc.) and full non-compliance, respectively denoted as y_1, y_2, and y_3, used as the training data of the model.

Step S32: a neural network model is trained using the training data, a result is output by adopting a Sigmoid activation function, different models are obtained by calculating macros of prediction data with the macros between 0 and 1 and continuously changing training parameters, and a prediction model with a highest macro value is finally selected as the compliance prediction model.

In the process of constructing the compliance prediction model, the feature vector V of the patient multi-source information is used as the input of the model, since V={v_1, v_2, v_3, v_4}, it is obtained that:

V={v_(1−1),v_(1−2), . . . ,v_(1−n),v_(2−1),v_(2−2), . . . ,v_(2−m),v_(3−1),v_(3−2), . . . ,v_(3−l),v_4}

It is defined that in hidden layers, L=3, representing that a neural network containing three hidden layers is selected, in the neural network, the layers are fully connected, a linear relation of connection of any neuron of a l^{th} layer and any neuron of a l+1^{th} layer is represented as z=Σ [symbol] w_l x_l+b (where, w represents a weight parameter weight, and b represents a bias term biase), each neuron represents one activation function, and the activation function is represented by adopting Sigmoid as σ(z)=1/(1+e\hat{ }(−z))=1/(1+e\hat{ }(−Σ [symbol] w_l x_l+b)). In output results of an output layer, an actual result is denoted as Y, a prediction result is denoted as [symbol] , and Y or [symbol] may be y_1, y_2, and y_3, respectively representing that the results are full compliance, partial compliance and full non-compliance. Due to a feature of the activation function, all the output results are mapped between (0, 1), therefore, y_1, y_2, and y_3 respectively output the result of (0, 1), and a maximum in the y_1, y_2, and y3 is selected as a prediction classification result, denoted as [symbol] [symbol] =max(y_1, y [symbol] _2, y_3). For example, a k^{th} patient obtains that outputs of y_1, y_2, and y_3 are respectively 0.3, 0.7 and 0.4 through the feature vector of the patient multi-source information and the trained compliance prediction model, [symbol] [symbol] =y [symbol] _2 is a prediction result, namely the prediction result of the k^{th} patient is partial compliance.

Through an above training method, the weight parameters W and the bias terms b corresponding to all the neurons in the model are solved. In the training process, medication taking behavior data of 1000 patients are randomly divided into 70% and 30%, 70% is used as a training set, and 30% is used as a test set. Since the model is a multi-classification model, macro F1 (denoted as macro-F1) is adopted as an evaluation index of the model. Firstly, distinguishing is divided into true positives (TPs), true negatives (TNs), false positives (FPs) and false negatives (FNs). TPs represent that positive samples are successfully predicted as positives; TNs represent that negative samples are successfully predicted as negatives; FPs represent that negative samples are falsely predicted as positives; and FNs represent that positive samples are falsely predicted as negatives. In the embodiment, prediction results are divided into three classifications, a h^{th} classification in the test set is used as a positive sample (where h may be 1, 2, and 3), a non-h^{th} classification is used as a negative sample, and a TP of the h^{th} classification is obtained, and denoted as a TPₕ; a TN of the h^{th} classification is denoted as a TNₕ; an FP of the h^{th} classification is denoted as an FPₕ; and an FN of the h^{th} classification is denoted as an FNₕ. According to a dichotomy accurate rate (denoted as P) formula P=TP/(TP+FP), an accuracy rate P_h=TP_h/(TP_h+FP_h) of the h^{th} classification is obtained; according to a recall rate (denoted as R) formula R=TP/(TP+FN) in a dichotomy, a recall rate R_h=TP_h/(TP_h+FN_h) of the h^{th} classification is obtained; and according to a balance F fraction (denoted as F) formula F=(2*P*R)/(P+R) in the dichotomy, a balance F fraction F_h=(2*P_h*R_h)/(P_h+R_h) of the h^{th} classification is obtained. According to a formula macro−F_1=1/Q Σ_(h=1)\hat{ }Q [symbol] F_h, since the embodiment is a three-way classification, Q=3. The quality of the model can be measured by calculating a macro F1 (macro-F1) of the prediction set, numbers of the macro F1 are between 0 and 1, the closer the numbers are to 1, the more accurate the model is, different models are trained by changing model parameters, and the prediction model with a highest macro F1 (nacro-F1) value is finally selected as the patient compliance prediction model.

Step S4: a patient category is predicted by using the compliance prediction model to obtain a patient classification; and
when there are new patient data generated or corrected patient data, after the feature vector V of the patient multi-source information used as the input of the prediction model passes through the patient compliance prediction model, the output [symbol] can be predicted as the patient classification, mainly being divided into full compliance, partial compliance and full non-compliance, and corresponding publicity-education pushing is performed on patients of partial compliance and full non-compliance.

Step S5: a system rule base is built by using the patient multi-source information, and after searching for a corresponding disease and treatment in the patient publicity-education knowledge graph through feedback information frim the system rule base, the disease and the treatment are pushed to the patient through the publicity-education applet;
step S51: the feature vector of the eye movement information in the patient multi-source information is used as an input, a state of the eye movement information is used as a feedback, rules are established through the input and the feedback, and the system rule base is formed by the plurality of rules;
the pushing system rule base is established, relevant information is searched for in

the patient publicity-education knowledge graph, and publicity-education pushing is performed on the patient through the publicity-education applet, so that the patient is changed into a patient of complete compliance from partial compliance or full non-compliance. When the pushing system rule base is established, the feature vector of the eye movement information that is v_3={v_(3−1), v_(3−2), . . . , v_(3−l)} in the patient multi-source information is mainly used as an “input” denoted as input in the rule base, according to a state of the eye movement information, a corresponding “return” denoted as return is given, so as to obtain required return information, the return information includes “content” denoted as content and a “form” denoted as form, and a storage form of an i^{th} rule is denoted as Rule_i={input: {v_(3−1): Input_1, v_(3−2): Input_2, . . . , v_(3−l): Input_n}, return{content: Return_1, form: Return_2}, where Input_1 . . . Input _n represent values which should be taken by the feature vectors in the rules, Return_1 and Return_2 respectively represent results of the content and the form returned when a value taken by a vector in the input is met. It is assumed that there are s rules stored in the rule base, the rule base may be represented as Ruleset=[Rule_1, Rule_2, . . . , Rule_s].

Step S52: the feature vector of the eye movement information is input to the system rule base, and after searching for the corresponding disease and treatment in the patient publicity-education knowledge graph through content and a form returned by the system rule base, content of an adverse reaction and a corresponding video obtained by searching are pushed to the patient through the publicity-education applet; and
after the return is received, searching is performed in the patient publicity-education knowledge graph through the content and the form of the return, during searching, the content is used as a basis of the predicate of the triad of <subject, predicate, object> in the patient publicity-education knowledge graph, and the return of the content may be adverse reactions, ingredients, postoperative complications, etc. and corresponds to the predicate in the patient publicity-education knowledge graph; after the content is determined, the form is searched for, and if form=“video” or form=“picture”, on the basis of the content, a predicate of the “picture” or the “video” is searched for. For example, one rule is {input:{“detention page content”: “adverse reactions”: “detention time”: “10 s”, return{content: “adverse reactions”, form: “yideo”}, representing that when it is detected that the “detention page content”=“adverse reactions” and “detention time”=“10 s” in the eye movement information of the patient, two results of content=“adverse reactions” and form=“video” will be returned, after searching for the corresponding disease and treatment in the patient publicity-education knowledge graph after combination with treatment, the “adverse reactions” are searched for to get the content of the adverse reactions, a video name is searched for through the “video”, a corresponding video name is searched for in a folder where the video is stored, and the video is pushed to the patient through the publicity-education applet.

In terms of the content of the automatic pushing system, the predicate of the patient publicity-education knowledge graph may be returned according to the patient information, and searching and pushing are performed through the patient publicity-education knowledge graph.

For example, the patients do not open or read the patient publicity-education knowledge graph pushed by the publicity-education apple, possibly due to not seeing the information, not knowing how to use a smart phone, not caring or not paying attention, etc., which should be solved through pushing or manual communication, and its importance should be explained at the same time; or, when the patients watch the patient publicity-education knowledge graph and it is found that eye movement information has collected more data on the page related to the adverse reactions, it is considered that the patients pay more attention to the adverse reactions, and pushing detailed information about the adverse reactions and other pushing modes are adopted, and predicate information of the patient publicity-education knowledge graph returned from the pushing rule base is adopted to get the content of the automatic pushing system.

Based on the content of the automatic pushing system, when it is found that the patients are more likely to accept the picture or video through the eye movement data, the automatic pushing system will further search for their pictures and videos for pushing based on the judgment of the content and the form of pushing. According to the collection of the eye movement data, the fixation time, the number of fixations, and a backward looking frequency are significantly higher than those of normal people, through an abnormal eye movement mode, a long average fixation time, a small average eye movement amplitude, a disorder saccade trajectory, and the lack of planning, strategy and organization of watching text, it is judged that the patients have “dyslexia”, for these patients, the pictures or videos are pushed, and the patients are easier to take in information.

The present disclosure applies the patient multi-source information fusion to the compliance prediction model. On the basis of the original electronic medical record feature, the patient personality inventory in the publicity-education applet and the eye movement tracking information when reading the publicity-education knowledge graph are considered to make patient portraits more three-dimensional and the compliance model more accurate. The patient publicity-education knowledge graph is constructed, and the publicity-education knowledge is visually shown in the way and structure of the publicity-education knowledge graph, and applied to the search during publicity education. The patient publicity knowledge graph integrates the forms of text, pictures and videos, searches for and pushes the required content more efficiently and quickly through entities and predicates, and identifies the best form of pushing through an eye movement tracking technology. A mode of combining knowledge with clinical data not only includes knowledge from guidelines, books, etc., but also carries out publicity-education pushing on the patients in combination with the actual clinical data; and when the patient multi-source information or patient behavior changes, patient classification and related publicity-education pushing will be corrected.

The above is only a preferred embodiment of the present disclosure and is not used to limit the present disclosure, and for those skilled in the art, the present disclosure may have various modifications and changes. Any modification, equivalent substitution, improvement, etc. made within the spirit and principles of the present disclosure shall be included in the scope of protection of the present disclosure.

What is claimed is:

1. A publicity-education pushing method based on a multi-source information fusion, comprising steps of:
step S1: constructing a patient publicity-education knowledge graph through public knowledge, a clinical expert supplement and an electronic medical record, and pushing the patient publicity-education knowledge graph to a patient through a publicity-education applet;
step S2: collecting patient basic information and patient diagnosis-treatment information through the electronic medical record, collecting patient eye movement information and a patient personality inventory through the publicity-education applet, and fusing and correcting the patient basic information, the patient diagnosis-treatment information, the patient eye movement information and the patient personality inventory to obtain patient multi-source information;
step S21: collecting the patient basic information and the patient diagnosis-treatment information through the electronic medical record, the patient basic information comprises fields corresponding to following parameters: a patient identity card number, a patient ID, an age, an education level, a geographical factor and/or accompanying status of family members, and the patient diagnosis-treatment information comprises fields corresponding to following parameters: visit datetime, the disease, a treatment mode, a drug treatment and/or a surgical treatment; and fusing the patient basic information and the patient diagnosis-treatment information to obtain electronic medical record information, with the electronic medical record information corresponding to a latest visit datetime as valid electronic medical record information;
step S22: sending, by the publicity-education applet, the patient publicity-education knowledge graph corresponding to the disease to the patient, collecting the patient eye movement information and the patient personality inventory through the publicity-education applet, and fusing the patient eye movement information and the patient personality inventory through the patient identity card number to obtain publicity-education applet information, with the publicity-education applet information corresponding to a latest video datetime as valid publicity-education applet information;
step S23: fusing the valid electronic medical record information and the valid publicity-education applet information through the patient identity card number to obtain the patient multi-source information;
step S24: identifying whether the electronic medical record information corresponding to the latest visit datetime in the electronic medical record information is consistent with electronic medical record information of the valid electronic medical record information, identifying whether the publicity-education applet information corresponding to the latest video datetime is consistent with publicity-education applet information of the valid publicity-education applet information; if at least one of the two identification results represents inconsistency, repeating step S21 to step S23 for re-fusion until both identification results represent consistency to complete a correction of the patient multi-source information;
step S3: constructing a compliance prediction model through a neural network by using the patient multi-source information and data collected on patient medication-taking behavior;
step S4: predicting a patient category by using the compliance prediction model to obtain a patient classification; and
step S5: building a system rule base by using the patient multi-source information, and after searching for a corresponding disease and treatment in the patient publicity-education knowledge graph through feedback information from the system rule base, pushing the disease and the treatment to the patient through the publicity-education applet.

2. The publicity-education pushing method based on a multi-source information fusion according to claim 1, wherein, in step S1:
the public knowledge is a patient treatment method, and an adverse reaction and/or an indication to the treatment method collected from a public knowledge base, an electronic version of guide and/or a disease form;
the clinical expert supplement is a supplement and refinement to incompleteness of the disease and/or incompleteness of a disease treatment mode performed through clinical experience of a clinician and/or a relevant expert; and
the electronic medical record comprises clinical data information of electronic medical records from a plurality of medical institutions.

3. The publicity-education pushing method based on a multi-source information fusion according to claim 1, wherein, in step S1, storage of the patient publicity-education knowledge graph represents a triad in the form of <subject, predicate, object> through an RDF structure, and finally the patient publicity-education knowledge graph with the disease as the subject, an adverse reaction, the surgical treatment and/or the drug treatment as the predicate, and a value pointed by the predicate as the object is formed; and a storage structure of the patient publicity-education knowledge graph is presented in a multimodal manner, comprising text, and a text-related picture and/or video.

4. The publicity-education pushing method based on a multi-source information fusion according to claim 1, wherein, in step S22, the patient eye movement information comprises detention page content, an average fixation time, a number of fixations, a fixation order, an average saccade amplitude, a number of saccades, a scanning duration, and/or a scanning direction; and the patient personality inventory comprises openness, conscientiousness, extraversion, agreeableness, emotional stability, and/or non-personality-inventory data without actively performing personality inventory.

5. The publicity-education pushing method based on a multi-source information fusion according to claim 1, wherein, step S3 comprises sub-steps of:
step S31: collecting the data on patient medication-taking behavior, the data on patient medication-taking behavior being divided into full compliance, partial compliance and full non-compliance, and using the patient multi-source information and the data on patient medication-taking behavior as training data for a model; and
step S32: training a neural network model using the training data, outputting a result by adopting a Sigmoid activation function, obtaining different models by calculating macros of prediction data with the macros between 0 and 1 and continuously changing training parameters, and finally selecting a prediction model with a highest macro value as the compliance prediction model.

6. The publicity-education pushing method based on a multi-source information fusion according to claim 1, wherein, step S5 comprises sub-steps of:
step S51: using a feature vector of the eye movement information in the patient multi-source information as an input and a state of the eye movement information as a feedback, establishing rules through the input and the feedback, and forming the system rule base by the plurality of rules; and
step S52: inputting the feature vector of the eye movement information to the system rule base, and after searching for the corresponding disease and treatment in the patient publicity-education knowledge graph through content and a form returned by the system rule base, pushing content of an adverse reaction and a corresponding video obtained by searching to the patient through the publicity-education applet.

7. The publicity-education pushing method based on a multi-source information fusion according to claim 3, wherein, in step S5, the predicate of the patient publicity-education knowledge graph is returned according to patient information, and searching and pushing are performed through the patient publicity-education knowledge graph.

8. The publicity-education pushing method based on a multi-source information fusion according to claim 1, wherein, in step S5, when it is found that the patient is more receptive to a picture or a video through eye movement data, further searching for pictures and videos of pushing content and a pushing form through a determine of an automatic pushing system on the pushing content and the pushing form, and pushing the pictures and videos.

9. A publicity-education pushing system based on a multi-source information fusion, comprising:
a patient publicity-education knowledge graph module, configured to push a patient publicity-education knowledge graph to a patient for publicity and education;
a patient multi-source information fusion module, configured to collect patient basic information and patient diagnosis-treatment information through an electronic medical record, collect patient eye movement information and a patient personality inventory through a publicity-education applet, and fuse and correct the patient basic information, the patient diagnosis-treatment information, the patient eye movement information and the patient personality inventory to obtain patient multi-source information;
a functional flow of the patient multi-source information fusion module is as follows:
step one: collecting the patient basic information and the patient diagnosis-treatment information through the electronic medical record, the patient basic information comprises fields corresponding to following parameters: a patient identity card number, a patient ID, an age, an education level, a geographical factor and/or accompanying status of family members, and the patient diagnosis-treatment information comprises fields corresponding to following parameters: visit datetime, the disease, a treatment mode, a drug treatment and/or a surgical treatment; and fusing the patient basic information and the patient diagnosis-treatment information to obtain electronic medical record information, with the electronic medical record information corresponding to a latest visit datetime as valid electronic medical record information;
step two: sending, by the publicity-education applet, the patient publicity-education knowledge graph corresponding to the disease to the patient, collecting the patient eye movement information and the patient personality inventory through the publicity-education applet, and fusing the patient eye movement information and the patient personality inventory through the patient identity card number to obtain publicity-education applet information, with the publicity-education applet information corresponding to a latest video datetime as valid publicity-education applet information;
step three: fusing the valid electronic medical record information and the valid publicity-education applet information through the patient identity card number to obtain the patient multi-source information; and
step four: identifying whether the electronic medical record information corresponding to the latest visit datetime in the electronic medical record information is consistent with the electronic medical record information of the valid electronic medical record information, identifying whether the publicity-education applet information corresponding to the latest video datetime is consistent with publicity-education applet information of the valid publicity-education applet information, if at least one of the two identification results represents inconsistency, repeating step one to step four for re-fusion until both identification results represent consistency to complete a correction of the patient multi-source information;
a compliance prediction module, configured to construct a compliance prediction model through a neural network by using the patient multi-source information and data collected on patient medication-taking behavior, and obtain a patient classification by a prediction through the compliance prediction model; and
an automatic pushing system module, configured to build a system rule base by using the patient multi-source information, and after searching for a corresponding disease and treatment in the patient publicity-education knowledge graph through feedback information from the system rule base, push the disease and the treatment to the patient through the publicity-education applet.
